(19) United States
(12) Patent Application Publication
(10) Pub. No.: US 2002/0119173 A1
(43) Pub. Date: Aug. 29, 2002

(54) Cosmetic compositions for preventing skin irritation

(76) Inventors: Samuel Qcheng Lin, Paramus, NJ (US); Ronni Lynn Weinkauf, Oradell, NJ (US); Uma Santhanam, Tenafly, NJ (US); David Roland Otts, Appleton, WI (US); Beth Anne Lange, Appleton, WI (US); David John Tyrrell, Appleton, WI (US); Chantel Spring Buhrow, Weyauwega, WI (US)

Correspondence Address: UNILEVER, PATENT DEPARTMENT, 45 RIVER ROAD, EDGEWATER, NJ, 07020, US

(21) Appl. No.: 09/886,154

(22) Filed: Jun. 21, 2001

Related U.S. Application Data
(60) Provisional application No. 60/257,879, filed on Dec. 22, 2000.

(51) Int. Cl. A61K 7/00; A61K 35/78

(52) U.S. Cl. 424/401; 424/729; 424/769; 424/737; 424/725

(57) ABSTRACT

A cosmetic composition is provided that includes petroleum jelly and an anti-irritant agent which achieves at least a 10% reduction of Interleukin-1 alpha in an EpiDerm™ Skin Culture Model. The agent may be a botanical active or a decoupling polymer. Particularly preferred botanicals are echinacea, yucca, green tea and willow herb.

### BACKGROUND OF THE INVENTION

1. Field of the Invention

The invention concerns compositions that cosmetically prevent skin irritation, especially irritant contact dermatitis, such as diaper rash caused by fecal enzymes.

2. The Related Art

There are many causes for skin irritation. Some derive from an abnormal functioning of the skin, and these are associated with disease conditions. Others are topically inflicted through contact with toxic plants, surfactants and other chemical ingredients of personal care or household products. Irritation may also arise from contact with fecal enzymes leading to a condition known as diaper rash. Irritants often operate by disrupting the skin's lipid/protein barrier. This barrier serves to prevent penetration of most substances to the lower viable layers of the skin, as well as preventing water loss.

Fecal enzyme contamination is a major source of irritation for large numbers of individuals. Infants in wet and/or soiled diapers are subject to the problem. Patients with colostomies and elderly adults suffering from incontinence may also experience the rash. There is a need to address the problem.

U.S. Pat. Nos. 5,869,033 and 5,702,709 (Schulz et al.) report control of diaper rash through incorporation of organophilic clays into the matrix of the diapers. U.S. Pat. No. 6,017,549 (Knight et al.) focuses on resolving irritation induced by contact with harsh emulsifiers or surfactants. Among suggested antidotes are alkyl polyosides, grafted water soluble proteins on a hydrophobic backbone, and lecithin.

Petroleum jelly, such as substances sold under the brand Vaseline® has long been known for its occlusive properties in preventing moisture loss and thereby healing damaged skin. Improvements in petroleum jelly have been reported in U.S. Pat. No. 5,552,148 (Znaiden et al.) disclosing formulations with inositol phosphates. In U.S. Pat. No. 5,552,147 (Znaiden et al.) petroleum jelly has been utilized as a vehicle for delivering alpha-hydroxy carboxylic acids as an anti-aging therapy. U.S. Pat. No. 5,595,745 (Znaiden et al.) discloses combination of behenoyl lactylates in petroleum jelly to achieve improved healing and moisturization.

It is an object of the present invention to improve upon the earlier technology by providing cosmetic compositions which prevent skin irritation.

Another object of the present invention is to provide a cosmetic composition capable of moisturizing and conditioning skin.

These and other objects of the present invention will become more readily apparent from consideration of the following summary and detailed description.

### SUMMARY OF THE INVENTION

A cosmetic composition is provided that includes:

(i) from about 0.1 to about 99% by weight of petroleum jelly; and

(ii) an anti-irritant agent which achieves at least a 10% reduction of Interleukin-1 alpha in an EpiDerm™ Skin Culture Model, the agent being selected from the group consisting of a botanical active extracted from a plant, decoupling polymers and mixtures thereof.

### DETAILED DESCRIPTION OF THE INVENTION

Now a highly efficient anti-irritant cosmetic composition has been found in the combination of petroleum jelly and certain anti-irritant agents which exhibit at least 10% reduction of Interleukin-1 alpha in an EpiDerm™ Test. These agents are either botanical actives or decoupling polymers, the latter being defined as synthetic polymers with a hydrophilic backbone and at least one hydrophobic side-chain.

Accordingly, a first element of compositions according to the present invention is that of petroleum jelly which is also known as petrolatum. Amounts of this material may range from about 0.1 to about 99%, preferably from about 10 to about 97%, more preferably from about 30% to about 99%, optimally from about 50 to about 95%, most especially from about 60 to about 90% by weight.

Anti-irritant agents according to the present invention are substances which achieve at least 10% reduction in Interleukin-1 alpha amounts in an EpiDerm™ Test. A detailed description of this test is provided under the Example section of the specification. EpiDerm™ is a multi-layer substrate of progressively differentiated keratinocytes, a cornified, air-interfaced human skin culture model that resembles normal human epidermis.

Botanicals are one class of anti-irritant agent suitable for the present invention. By the term “botanicals” is meant any water soluble or oil soluble active extracted from a particular plant. Suitable botanicals are actives which are extracted from echinacea, yucca glauca, willow herb, basal leave, bell pepper, black tea, blackberry, black currant fruit, coffee seed, dandelion root, date palm fruit, gingko leaf, green tea polyphenols (i.e. including epicatechin gallate and epigallocatechin 3-O-gallate), hawthorn berries, licorice, sage, strawberry, sweet pea, tomato, vanilla fruit, neohesperidin, rutin, morin, myricetin, chlorogenic acid glutathione and any combinations thereof. Most preferred are echinacea, yucca glauca, green tea and willow herb. Echinacea actives may be obtained from the following echinacea species: Echinacea angustifolia, Echinacea purpurea, Echinacea pallida.

Amounts of the botanicals in terms of active component (no solvent) may range from about 0.000001 to about 10%, preferably from about 0.00001 to about 5%, more preferably from about 0.0001 to about 1%, optimally from about 0.0001 to about 0.5%, but more preferably from about 0.001 to about 0. 1% by weight.

Decoupling polymers may also be effective as the anti-irritant agent. Preferred are acrylic polymers having a hydrophilic backbone and at least one hydrophobic side-chain. The hydrophilic backbone of the decoupling polymer is preferably composed of one or two monomer types but also possible is the use of three or more different monomer types in one hydrophilic backbone. Examples of preferred hydrophilic backbones are: homopolymers of acrylic acid, copolymers of acrylic acid and maleic acid, poly(2-hydroxy ethyl acrylate), polysaccharides, cellulose ethers, polyglycerols, polyacrylamides, polyvinylalcohol/polyvinylether copolymers, poly(sodium vinyl sulphonate), poly(2-sulphato ethyl methacrylate) and poly(acrylamidomethylpropane sulphonate).

Preferably the hydrophobic side chains are part of a monomer unit which is incorporated in the polymer by copolymerizing hydrophobic monomers and the hydrophilic monomers making up the backbone of the polymer. The hydrophobic side chains for this use preferably include those which when isolated from their linkage are relatively water insoluble, i.e., preferably less than 1 g/l, more preferably less than 0.5 g/l, optimally less than 0.1 g/l of the hydrophobic monomers will dissolve in water at ambient temperature and a pH of 3.0 to 12.5.

Preferably the hydrophobic moieties are selected from siloxanes, saturated and unsaturated alkyl chains, e.g. having from 5 to 24 carbon atoms, preferably from 6 to 18, optimally from 8 to 16 carbon atoms, and are optionally bonded to the hydrophilic backbone via an alkoxylene or polyalkoxylene linkage, for example a polyethoxy, polypropoxy or butyloxy (or mixtures of same) linkage having from 1 to 50 alkoxylene groups. Alternatively the hydrophobic side chain may be composed of relatively hydrophobic alkoxy groups, for example butylene oxide and/or propylene oxide, in the side-chain(s) and will essentially have the character of a nonionic surfactant. Specific examples of the anti-irritant agent polymers may be found in U.S. Pat. No. 5,147,576 (Montague et al.) herein incorporated by reference. Amounts of the polymer may range from about 0.1 to about 20%, preferably from about 0.5 to about 10%, optimally from about 1 to about 5% by weight.

A variety of inorganic water-insoluble materials may be employed to boost effectiveness of the anti-irritant agents. These boosters may be selected from a wide variety of natural or synthetic clays and zinc oxides. Among the useful clays are montmorillonite, bentonite, beidellite, hectorite, saponite and stevensite. Particularly useful are organophilic clays which are prepared from the aforementioned natural or synthetic clays and treated with quaternary ammonium compounds. Normally the quaternary ammonium compounds are quaternized amines having one or two C₁₄-C₂₀ chain substituents and two or three C₁-C₄ short chain substituents (e.g. methyl groups). Particularly preferred is dimethyl dihydrogenated tallow ammonium salts, which are available as quaternium 18 bentonite and quaternium 18 hectorite. Amounts of the booster may range from about 0.5 to about 15%, preferably from about 3 to about 8% and optimally about 5% by weight.

Although compositions according to the present invention may be anhydrous, they usually will contain water in amounts from 0 to about 15%, preferably from about 0.8 to about 10%, optimally from about 1 to about 8%, especially from about 4 to about 6% by weight.

Beyond the aforementioned components, the present invention may also include other ingredients typically found in cosmetic formulations. Among these ingredients are emollients, humectants, thickeners, preservatives, fragrances and vitamins.

Emollients may be selected from materials such as C₈-C₃₀ fatty alcohols, triglyceride oils, silicone oils and a variety of esters. Amounts of the emollients may range from about 0.5 to about 20%, preferably from about 1 to about 10%, optimally from about 2 to about 8% by weight. Illustrative emollients are stearyl alcohol, cetyl alcohol, isopropyl palmitate, isopropyl myristate, lanolin, sunflower oil, evening primrose oil, soybean oil, dimethicone, cyclomethicone, dimethicone copolyol and dimethyl polysiloxane.

Among the useful preservatives are methyl paraben, propyl paraben, EDTA salts, potassium sorbate, potassium benzoate and DMDM hydantoin.

Cosmetic compositions of the present invention may also contain vitamin ingredients such as Vitamin A palmitate, Vitamin E acetate, Niacin (Niacinamide), Vitamin C and combinations thereof.

Emulsifiers, particularly those of HLB below 7, may also be useful for purposes of the present invention at levels to from about 0.1 to about 10% by weight. These emulsifiers may be alkoxylated C₈-C₃₀ fatty acids and fatty alcohols. Examples of such materials are polyoxyethylene (2) lauryl ether, polyoxyethylene (3) monostearate, polyoxyethylene (6) cetyl ether and polyoxyethylene (5) stearyl ether and Myreth-3-Myristate (CTFA name) available commercially as Cetiol 1414-E®. Other suitable emulsifiers included cetyl phosphate salts and dimethicone copolyol, the latter commercially available as ABIL®EM90 from Goldschmidt AG. Phosphatides such as lecithin may also be useful as emulsifiers in these systems.

Except in the operating and comparative examples, or where otherwise explicitly indicated, all numbers in this description indicating amounts of material ought to be understood as modified by the word “about”.

The following examples will more fully illustrate the embodiments of this invention. All parts, percentages and proportions referred to herein and in the appended claims are by weight unless otherwise illustrated.

### EXAMPLE 1

There appear to be multiple factors contributing to the clinical manifestations of diaper rash. These include increases in skin hydration due to the occlusive nature of the diaper as well as repeated skin contact with urine and feces. When the surface of diapered skin is compromised, as a result of excessive hydration and/or physical injury following wiping, fecal and urine irritants have a greater chance of penetrating through the stratum corneum and reacting with the underlying viable keratinocytes. In the complex milieu of irritants found in feces and urine there are a variety of enzymes (proteases, lipases and glycosidases) from the host and bacteria. Keratinocytes exposed to the protease trypsin at nanomolar concentrations upmodulate the production of multi-functional inflammatory mediators such as interleukin-8 and granulocyte-macrophate colony-stimulating factor. Trypsin has been proposed to transiently disrupt cell membranes providing exit for immunoregulatory proteins that do not contain leader sequences and signaling peptides such as interleukin-1 alpha (IL-1 alpha).

Studies performed with EpiDerm™ Skin Culture model have shown that purified trypsin or fecal extract with high trypsin activity will upregulate in a time-and dose-dependent fashion the expression of IL-1 alpha in the underlying media. Interleukin-1 alpha is one of the primary initiators of cutaneous inflammation activating a number of cells (endothelial cells, fibroblasts, and keratinocytes) to synthesize an array of cytokines that induce rapid physiological changes. Such alterations in cell function can potentially lead to clinical signs of diaper rash (these can include erythema and swelling).

Materials

The fecal protease mixture insult was prepared by diluting a 10 mg/ml stock (50 mM NaOAc, pH 5.5, 0.15M NaCl stored at -80° C.) in PBS to a working concentration of 250 ug/ml. One milliliter of the stock protease insult solution contains 2558 USP units of trypsin and 298 USP units of chymotrypsin and is available from Specialty Enzymes, Inc., Chino, Calif. The bile acid insult was prepared fresh by dissolving 65 mg of cholic acid, 62 mg of deoxycholic acid, and 31 mg of chenodeoxycholic in 10 ml of PBS. Components of the bile acid stock were purchased from Sigma Chemical Co., St. Louis, Mo. Phosphate-buffered saline, pH 7.4 (PBS) was purchased from Life Technologies, Gaithersburg, Md. EpiDerm™ EPI-200 Skin Culture model and the MTT kits (MTT-100) were purchased from MatTek Corp., Ashland, Mass.

In Vitro Measurement of Reduction of IL-1 alpha in EpiDerm™

EpiDerm™ inserts were added to six well plates containing one ml of pre-warmed media and acclimated in a 37° C., 5% CO₂ incubator for 30 minutes. The treatment or control (15 microliter) is then applied to the skin surface after removing any residual media. For test compositions using a petrolatum base, the composition is applied using a positive displacement pipettor and spread over the skin culture using a glass rod. Samples were incubated in the 37° C., 5% CO₂ incubator for 30 minutes; the underlying media was removed and replaced with fresh, pre-warmed media. The fecal protease insult (10 microliter) was then applied to the surface of the skin. After a 6 hour incubation in the incubator, the underlying media was removed and stored at −80° C. The following day the amount of IL-1 alpha was determined from each of the samples using the Interleukin-1 alpha Quantikine Kit (R&D Systems, Minneapolis, Minn.).

Interleukin-1 alpha measurements were converted to Log 10 for each of the treatments and the averages for each determined. To determine the ability of the formulated excipient compositions to reduce skin irritation induced by the protease insult, the percent mean reduction of IL-1 alpha was calculated as follows.

Percent mean reduction of IL-1 alpha=100* ((PJ control+protease)−(formulated excipient composition+protease)) divided by ((PJ control+protease)−(PJ control+PBS)). (Formulated excipient composition+protease)=the measured amount of IL-1 alpha from treatments with a complete PJ formulation (PJ+excipients+emulsifiers+other components as listed in Table 1) with protease insult. (PJ control+protease)=the measured amount of IL-1 alpha from a treatment with a partial PJ formulation without excipients with protease insult. (PJ control+PBS)=measured amount of IL-1 alpha from a treatment with a partial PJ formulation without excipients with PBS. The higher the reduction, the more effective the treatments (excipients) are in reducing irritation caused by the fecal protease insult.

Effect of Chemistries on Cell Viability Using the MTT Assay

The MTT assay is performed to ensure that the reduction in the amount of IL-1 alpha is not due to cell death. After removing the media, the EpiDerm™ insert was washed by consecutively immersing it in three different beakers of PBS (fresh PBS for each chemistry) and discarding the PBS on paper towel. EpiDerm™ insert was patted onto paper towel and then placed into wells of a 24 well plate containing 300 microliter of pre-warmed media. After all the inserts were washed they were transferred to new 24 well plates containing 300 microliter of the MTT (Thiazoyl blue, (3-[4,5-Dimethylthiazol-2-yl]-2,5-diphenyltetrazolium bromide) reagent.

Following incubation of the tissue for 2 hrs. in a 37° C., 5% CO₂ incubator, the EpiDerm™ inserts were then transferred to 24 well plates and immersed in 2 ml of MTT extraction buffer (MatTek Corporation). These plates were parafilmed, covered and placed in a photec bag to reduce evaporation of the extraction buffer. After rocking the plates overnight in the dark, the liquid in the inserts was decanted back into the wells. Samples were mixed and a 200 microliter aliquot removed from each well and transferred to a 96 well plate. The optical density (OD) of the samples was measured at 570 nm. This reading was subtracted from a background reading at 650 nm to improve the quality of the data. Percent viability was calculated as 100×(Mean OD sample/Mean OD PBS control).

TABLE I
Cosmetic Compositions for Reducing Skin Irritation

| | Composition (Weight %) | | | | | |
|---|---|---|---|---|---|---|
| Ingredients | A | B | C | D | E | F |
| Petroleum Jelly | 82.2 | 82.2 | 73.2 | 73.2 | 80.72 | 80 |
| Sunflower Oil | 10 | 10 | 10 | 10 | — | — |
| Glycerin | 5 | 5 | 5 | 5 | — | — |
| Soy sterol | 0.8 | 0.8 | 0.8 | 0.8 | — | — |
| Prolipid ® 141 | 1 | 1 | 1 | 1 | — | — |
| Abil ® EM90 | — | — | — | — | 1.68 | — |
| Echinacea | — | — | — | — | 16.8 | — |
| Extract | | | | | | |
| Yucca Extract | — | 1 | — | 10 | — | — |
| Willow Herb | 1 | — | 10 | — | — | — |
| Extract | | | | | | |
| Narlex ® DC-1 | — | — | — | — | — | 20 |
| (10% solids in | | | | | | |
| water) | | | | | | |
| Water | — | — | — | — | — | — |
| Preservatives | — | — | — | — | 0.8 | — |

Table II summarizes the test results. They reveal that all cosmetic compositions were effective in reducing skin irritation in the skin construct, EpiDerm™. Based on their efficacy in the in vitro skin model, these petrolatum-based formulations containing the cosmetic compositions were evaluated for their ability to mitigate skin irritation in an adult back dermatitis clinical.

TABLE II
Effect of Cosmetic Compositions on Reducing Skin Irritation

| | Mean reduction | |
|---|---|---|
| Composition | Interleukin-1 alpha, % | Viability, 10% |
| A | 41; 36 | 92; 93 |
| B | 30; 26 | 88; 85 |
| C | 16; 44 | 87, 94 |
| D | 24; 14 | 93; 85 |
| E | 18 | 99 |
| F | 53 | 101 |

Mean reduction of the inflammatory marker (IL-1 alpha) for two experiments are shown (A, B, C and D). Mean reduction of the inflammatory marker (IL-1 alpha) for a single experiment is shown (E and F). In all cases, five replicates for each of the cosmetic compositions were performed.

### EXAMPLE 2

Suitable cosmetic compositions were further subjected to clinical test on adult backs. The compositions evaluated were: 0.2% to 16.8% of actives such as yucca, willow herb, echinacea, or Narlex® DC-1; 0% to 1.68% Abil® EM90 (dimethicone copolyol); 0.8% preservatives and/or stabilizers (propyl paraben, methyl paraben, disodium EDTA, BHT, and NaCl); and balance to 100% petrolatum jelly. Narlex® DC-1 as employed contained 34% solids. The two controls were petroleum jelly containing the same amounts of Abil® EM90 and propyl paraben and 100% irritant mixture, respectively.

Clinical Protocol

1. Panel size was a minimum of 17 adult males and females. Up to 16 sites per adult back were employed for the experiment. Each site was 2.5 cm in diameter.

2. The irritant mixture included trypsin-chymotrypsin and bile acid in PBS at a concentration of 1500 ug/ml. It was freshly prepared or refrigerated at−80° C. and defrosted at 37° C. just prior to use and held in an ice bath. In each treatment, 0.2 ml was placed into a 25 mm Hill Top Chamber.

3. The grading system was a scoring scale combining erythema and edema on a 0 - 4 scale with ½grades.

4. Samples of 30 mg were applied to the subject's back site for 20 minutes before application of the insult.

5. The Hill Top Chamber with irritant was taped onto the site for 24 hrs.

6. After the 24 hrs treatment, experts examined the test sites 30 minutes after patch removal and recorded the results.

7. Data analysis was preformed using Nonparametic Wilcoxon signed rank test statistical treatment of data for significant difference between two treatments.

The lower the erythema score, the more effective the treatments. Table III reveals that petroleum jelly (PJ) and 16.8% botanical extracts treatments began evidencing lower erythema scores after 7 days' treatments. These results were much improved over both the irritant mixture and petroleum jelly. Tables IV and V report the results of Echinacea and Narlex® DC-1 polymers over a wide range of concentrations. Both treatments were effective over the tested concentration range. Echinacea is effective even as low as 0.2% concentration of the extract which is equivalent to about 0.0015% active botanical.

TABLE III
Skin Irritation/Rash Reduction with PJ
and Various Botanical Extracts (16.8%)

| | | | | PJ + | |
|---|---|---|---|---|---|
| | | | | surfactant | Irritant |
| Days | Echinacea | Yucca | Willow | control | control |
| 1 | 0.1 | 0.1 | 0.1 | 0 | 0.1 |
| 2 | 0.5 | 0.3 | 0.3 | 0.6 | 0.7 |
| 3 | 0.6 # | 0.5 # | 0.5 # | 0.6 # | 1.1 |
| 4 | 0.8 # | 1.1 | 0.8 # | 0.9 # | 1.5 |
| 5 | 1.0 # | 1.1 # | 1.0 # | 1.1 | 1.6 |
| 6 | 1.1 # | 1.4 # | 1.2 # | 1.4 # | 1.9 |
| 7 | 1.2 # | 1.3 # | 1.4 # | 1.4 # | 1.9 |
| 8 | 1.6 # | 1.4 # | 1.7 # | 1.9 # | 2.2 |
| 9 | 1.8 # | 1.7 # | 2.0 # | 2.1 # | 2.3 |
| 10 | 1.8 # | 1.9 # | 2.0 # | 2.2 # | 2.2 |

# Indicates significant differences from Irritant control at 95% confidence level

TABLE IV
Skin Irritation/Rash Reduction with PJ
and Echinacea at Various Concentrations

| | | | | | PJ + | |
|---|---|---|---|---|---|---|
| | | | | | surfactant | Irritant |
| Days | 0.2% | 1.7% | 7.5% | 16.8% | control | control |
| 1 | 0.21 # | 0.1 # | 0.3 # | 0.2 # | 0.1 | 0.6 |
| 2 | 0.3 # | 0.1 # | 0.3 # | 0.4 # | 0.2 | 0.9 |
| 3 | 0.6 # | 0.3 # | 0.6 | 0.7 | 0.5 | 1.2 |
| 4 | 0.8 # | 0.5 # | 0.3 # | 0.9 # | 0.6 | 1.7 |
| 5 | 1.0 # | 1.0* # | 1.2 # | 1.3 # | 1.4 | 1.9 |
| 6 | 1.4 # | 1.3 # | 1.4 # | 1.4 # | 1.7 | 2.1 |
| 7 | 1.6 # | 1.5 # | 1.6 # | 1.8 # | 2.0 | 2.1 |
| 8 | 1.6 # | 1.2* # | 1.8 # | 1.6 # | 2.0 | 2.1 |
| 9 | 1.6 # | 1.1 # | 1.9 # | 1.6 # | 1.8 | 2.3 |
| 10 | 1.5 # | 1.4* # | 2.0 # | 1.7 # | 1.9 | 2.4 |

*Indicates significant differences from PJ/Surfactant control at 95% confidence level
# Indicates significant differences from Irritant control at 95% confidence level

TABLE V
Skin Irritation/Rash Reduction with PJ
and Narlex ® at Various Concentrations

| | Concentration (Weight %) | | | | | |
|---|---|---|---|---|---|---|
| | | | | | PJ + | |
| | | | | | Surfactant | |
| Days | 16.8% | 7.5% | 1.7% | 0.2% | Control | Irritant |
| 1 | 0.9 # | 0.3 # | 0.2 # | 0.2 # | 0.1 | 0.6 |
| 2 | 0.2 # | 0.1 # | 0.4 | 0.4 | 0.2 | 0.9 |
| 3 | 0.2 # | 0.3 # | 0.7 | 0.6 | 0.5 | 1.2 |
| 4 | 0.6 # | 0.5 # | 0.8 #* | 1.0 # | 0.6 | 1.7 |
| 5 | 1.4 # | 1.0 # | 1.3 # | 1.5 # | 1.4 | 1.9 |
| 6 | 1.6 # | 1.0 #* | 1.6 # | 1.6 # | 1.7 | 2.1 |
| 7 | 1.7 # | 1.2 #* | 1.4 # | 1.5 # | 2.0 | 2.0 |
| 8 | 1.5 # | 1.1 #* | 1.6 # | 1.5 # | 2.0 | 2.0 |
| 9 | 1.5 # | 1.2 #* | 1.7 # | 1.6 # | 1.8 | 2.3 |
| 10 | 1.7 # | 1.5 #* | 1.7 # | 1.8 # | 1.9 | 2.4 |

*Indicates significant differences from PJ/Surfactant control at 95% confidence level
# Indicates significant differences from Irritant control at 95% confidence level

### EXAMPLE 3

A cosmetic composition suitable for the present invention is detailed in Table VI.

TABLE VI

| INGREDIENT | COMPOSITION (WEIGHT %) |
|---|---|
| Petroleum Jelly | 77.7 |
| Quaternium 18 Bentonite | 8 |
| Zinc Oxide | 5 |
| Cetiol ® 1414-E | 2 |
| Cholesterol | 2 |
| Lecithin | 2 |
| Behenyl Alcohol | 2 |
| Echinacea Extract (1% Active) | 1 |
| Abil ® EM90 | 0.1 |
| Disodium EDTA | 0.1 |
| BHT | 0.1 |

### EXAMPLE 4

Another cosmetic composition suitable for the present invention is detailed in Table VII.

TABLE VII

| INGREDIENT | COMPOSITION (WEIGHT %) |
|---|---|
| Petroleum Jelly | 71.3 |
| Zinc Oxide | 10 |
| Potassium Lactate | 5 |
| Cetiol ® 1414-E | 2 |
| Cholesterol | 2 |
| Lecithin | 2 |
| Stearic Acid | 2 |
| Yucca Glauca Extract (1% Active) | 5 |
| Abil ® EM90 | 0.5 |
| Disodium EDTA | 0.1 |
| BHT | 0.1 |

### EXAMPLE 5

Still another cosmetic composition suitable for the present invention is detailed in Table VIII.

TABLE VIII

| INGREDIENT | COMPOSITION (WEIGHT %) |
|---|---|
| Petroleum Jelly | 53.6 |
| Dimethicone | 25 |
| Quaternium 18 Bentonite | 5 |
| Zinc Oxide | 5 |
| Glycerol Monostearate | 2 |
| Cholesterol | 2 |
| Lecithin | 2 |
| Stearic Acid | 2 |
| Willow Herb Extract (10% Active) | 1 |
| Abil ® EM90 | 0.2 |
| Disodium EDTA | 0.1 |
| BHT | 0.1 |

### EXAMPLE 6

Yet another cosmetic composition suitable for the present invention is detailed in Table IX.

TABLE IX

| INGREDIENT | COMPOSITION (WEIGHT %) |
|---|---|
| Petroleum Jelly | 67.7 |
| Stearyl Alcohol | 10 |
| Zinc Oxide | 10 |
| Polyoxyethylene 20 Stearyl Ether | 2 |
| Soya Sterol | 2 |
| Lecithin | 1 |
| Green Tea Extract (10% Active) | 2 |
| Dimethicone | 5 |
| Abil ® EM90 | 0.1 |
| Methyl Paraben | 0.1 |
| Propyl Paraben | 0.1 |

### EXAMPLE 7

A further cosmetic composition suitable for the present invention is detailed in Table X.

TABLE X

| INGREDIENT | COMPOSITION (WEIGHT %) |
|---|---|
| Petroleum Jelly | 60.8 |
| Bentonite Clay | 5 |
| Zinc Oxide | 5 |
| Polyoxyethylene 20 Stearyl Ether | 2 |
| Cholesterol | 2 |
| Lecithin | 2 |
| Stearyl Alcohol | 2 |
| Narlex ® DC-1 (10% Active) | 10 |
| Dow Corning ® 245 | 10 |
| Abil ® EM90 | 1 |
| Methyl Paraben | 0.1 |
| Propyl Paraben | 0.1 |

The foregoing description and examples illustrate selected embodiments of the present invention. In light thereof variations and modifications will be suggested to one skilled in the art, all of which are within the spirit and purview of this invention.

What is claims is:

1. A cosmetic composition comprising:
(i) from about 0.1 to about 99% by weight of petroleum jelly; and
(ii) an anti-irritant agent which achieves at least a 10% reduction of Interleukin-1 alpha in an EpiDerm™ Skin Culture Model, the agent being selected from the group consisting of extracted botanical actives, decoupling polymers and mixtures thereof.

2. The composition according to claim 1 wherein the anti-irritant agent is a botanical active extracted from a plant selected from the group consisting of echinacea, yucca and willow herb.

3. The composition according to claim 1 wherein the anti-irritant agent is a botanical active extracted from green tea.

4. The composition according to claim 1 wherein the decoupling polymers have a hydrophilic backbone selected from the group consisting of homopolymers of acrylic acid, copolymers of acrylic acid and maleic acid, poly(2-hydroxyethylacrylate), polysaccharides, cellulose ethers, polyglycerols, polyacrylamides, polyvinyl alcohol/polyvinyl ether copolymers, poly(sodium vinyl sulfonate), poly(2-sulphato ethyl methacrylate) and poly(acrylamidomethyl propane sulphonate).

5. The composition according to claim 1 wherein the petroleum jelly is present in an amount from about 30% to about 99% by weight.

6. The composition according to claim 1 wherein water is present in an amount from 0 to 15% by weight.

7. The composition according to claim 1 where the anti-irritant agent is present in an amount from about 0.000001 to about 10% by weight.

8. The composition according to claim 1 further comprising an anti-irritant booster selected from the group consisting of clays, zinc oxide and mixtures thereof, present in an amount from about 0.5 to about 10% by weight.

9. A method for preventing skin irritation comprising applying to the skin a cosmetic composition comprising:
(i) from about 0.1 to about 99% by weight of petroleum jelly; and
(ii) an anti-irritant agent which achieves at least a 10% reduction of Interleukin-1 alpha in an EpiDerm™ Skin Culture Model, the agent being selected from the group consisting of extracted botanical actives, decoupling polymers and mixtures thereof.

10. The method according to claim 1 wherein the anti-irritant agent is a botanical extracted from a plant selected from the group consisting of echinacea, yucca, green tea and willow herb.
